(19) United States
(12) Patent Application Publication
(10) Pub. No.: US 2024/0226063 A1
(43) Pub. Date: Jul. 11, 2024

(54) SOLUBLE ANALOGUES OF 6BIO THEREOF AND IMPLEMENTATION THEREOF

(71) Applicant: JAWAHARLAL NEHRU CENTRE FOR ADVANCED SCIENTIFIC RESEARCH, Jakkur, Bangalore (IN)

(72) Inventors: James Premdoss Clement CHELLIAH, Jakkur, Karnataka Bangalore (IN); Ravi MANJITHAYA, Jakkur, Karnataka Bangalore (IN); Sridhar RAJARAM, Jakkur, Karnataka Bangalore (IN); Vijaya VERMA, Jakkur, Karnataka Bangalore (IN); Kavita SHARMA, Jakkur, Karnataka Bangalore (IN); Suresh Santhi NATESAN, Jakkur, Karnataka Bangalore (IN)

(21) Appl. No.: 18/557,822

(22) PCT Filed: Apr. 29, 2022
(86) PCT No.: PCT/IN2022/050408

§ 371 Date: Oct. 27, 2023

(30) Foreign Application Priority Data
Apr. 29, 2021 (IN) 202141019767

(51) Int. Cl. A61K 31/404; A61P 25/12; A61P 25/22; A61P 25/28

(52) U.S. Cl. CPC A61K 31/404; A61P 25/12; A61P 25/22; A61P 25/28

(57) ABSTRACT

The present disclosure discloses a composition comprising (2′Z, 3′E)-6-Bromoindirubin-3′-oxime or its derivative for use in treatment of neurodevelopmental and of neurodegenerative disorders. The disclosure further relates to a method for correcting the behavioural deficits in a subject with neurodevelopmental and neurodegenerative disorders comprising administering the subject with a therapeutically effective amount of (2′Z, 3′E)-6-Bromoindirubin-3′-oxime or its derivative.

### FIELD OF INVENTION

The present disclosure broadly relates to the field of neuroscience, and particularly refers to the use of soluble analogues of 6-BIO ((2′Z, 3′E)-6-Bromoindirubin-3′-oxime) and derivatives thereof for the treatment of neurodevelopmental and neurodegenerative disorders.

### BACKGROUND OF INVENTION

Everyday activities ranging from walking to computation is regulated by synaptic connections in the brain. Synaptic plasticity represents one of the most primary and significant functions of the brain, which is the ability of the neural activity generated by an experience to modify neural circuit function and thereby modify subsequent thoughts, feelings, and behaviour. This orchestrated brain activity requires several genes and proteins which are important for synaptic function. If any of these genes are mutated, the proteins expressed by the mutated genes may be non-functional and result in the loss of synaptic plasticity. This loss of synaptic plasticity subsequently causes several neurological disorders. Neurological disorders or disorders of the nervous system can be classified as neurodegenerative and neurodevelopmental disorders (Watson, et al., 2019, Omega-3 fatty acids in Brain and neurological health, Chapter 13).

Neurodegenerative diseases occur when nerve cells in the brain or peripheral nervous system lose function over time and eventually die. Alzheimer's disease and Parkinson's disease are the most common neurodegenerative diseases. In 2016, an estimated 5.4 million Americans were living with Alzheimer's disease and Parkinson disease that affects 1% of the ageing population. Although treatments may help relieve some of the physical or mental symptoms associated with neurodegenerative diseases, it is currently impervious to slow disease progression and no known cures. (National Institute of Environmental Health Sciences. 2021. Neurodegenerative Diseases. [online] Available at: <https://www.niehs.nih.gov/research/supported/health/neurodegenerative/index.cf m#:˜:text=Grant%20recipients%20study%20the%20following%20types%20of%20environmental,factors%20(e.g.,%20caffeine,%20tobacco%20smoke,%20dietary%2 0antioxidants>[Accessed 24 Feb. 2021]).

Neurodevelopmental disorders afflict patients under the age of 10, however, the difficulties caused by the disorder may persist well into adulthood. Most common neurodevelopmental disorders include attention deficit hyperactivity disorder (ADHD), autism spectrum disorder, dyslexia, and intellectual disability. The prevalence of autistic spectrum disorders across most countries is roughly 3-4 percent of the total population; this ranges from 2 to 4 percent across countries. People with intellectual disability experience deficits in intellectual and adaptive functioning which often comorbid with autism spectrum disorder traits which generally begin before adulthood. (Thapar et al., 2017. Neurodevelopmental disorders. The Lancet Psychiatry, 4(4), pp. 339-346.) and (Our World in Data. 2021. Neurodevelopmental disorders. [online] Available at: <https://ourworldindata.org/neurodevelopmental-disorders>[Accessed 24 Feb. 2021])

Currently, there is no cure for neurodevelopmental and neurodegenerative disorders and the treatment options for such neurological disorders is limited. For instance, the conventional treatments that are available for treating neurological disorders include “Psychobiotics” which are a group of probiotics that affect the central nervous system (CNS) related functions mediated by the gut-brain-axis via the immune, humoral, neural, and metabolic pathways to improve not only the gastrointestinal (GI) function but also the antidepressant and anxiolytic capacity. Other options require chronic rehabilitation that includes physiotherapy, pain management and pharmaceutical intervention. (Cheng et al., 2019. Psychobiotics in mental health, neurodegenerative and neurodevelopmental disorders. Journal of Food and Drug Analysis, 27(3), pp. 632-648)

The patent, U.S. Pat. No. 8,209,018B2 relates to the possible approaches to patient evaluation, warning about and the treatment of a neurological disorder. The patent document relies on historical data of the patient in short and/or long timescales obtained, that may be intermittent or temporally discontinuous from each other or other events of interest pertaining to seizures and the patient may thereby be treated based on the determined probability information.

The Patent Application, GB2503187A discloses pharmaceutical compositions, medicaments, and methods for use in preventing, ameliorating or treating neurodevelopmental disorders such as Autism spectrum disorder, intellectual disability, epilepsy, and including schizophrenia. The disclosed pharmaceutical composition targets dopamine and noradrenaline signalling for treating cognitive dysfunction.

The U.S. Pat. No. 9,486,472B2 relates to methods and reagents for modulating neuronal apoptosis or synaptic plasticity. The disclosed patent Application includes methods implemented by the administration of peptide inhibitors of alpha-Amino-3-hydroxy-5-methyl-4-isoxazoleproprionic acid (AMPA) receptor endocytosis.

Since there are limited options for the treatment of such neurological disorders, therefore, there is a dire need in the art to provide safe and effective methods of treatment of neurological disorders without any adverse effects.

### SUMMARY OF THE INVENTION

In an aspect of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative.

In another aspect of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative.

In another aspect of the present disclosure, there is provided with a composition comprising (2′Z, 3′E)-6-Bromoindirubin-3′-oxime or its derivative for the treatment of neurodevelopmental disorders and at least one excipient.

In another aspect of the present disclosure, there is provided with a composition comprising (2′Z, 3′E)-6-Bromoindirubin-3′-oxime or its derivative for the treatment of neurodegenerative disorders and at least one excipient.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the intraperitoneal injection regime for 6-BIO at a concentration of 5 mg/kg based on the weight of the mice and the age group segregation, in accordance with an embodiment of the present disclosure.

FIG. 2 depicts the effect of 6-BIO on hyperactivity or motor function of the wild type and Syngap1^{+/-} mice. FIGS. 2 (A), (B) and (C) depicts the total distance travelled by the wild type and Syngap1^{+/-} mice in open field chamber was grouped into 2(A) P10-16, the critical developmental period, 2(B) P10-80, critical period till adulthood, and 2(C) P30-80, adulthood and shown as individual data points. In FIG. 2(A) for P10-80 where behaviour was exhibited by the mice at P16 in the wild type (WT)-Vehicle: N=20; Syngap1^{+/-}-Vehicle: N=11; WT-6BIO: N=16; Syngap1^{+/-}-6BIO: N=14, F (1, 57)=14.83, p=0.0003, P10-80 where behaviour was exhibited at P42 (B), WT-Vehicle: N=17; Syngap1^{+/-}-Vehicle: N=9; WT-6BIO: N=15; Syngap1^{+/-}-6BIO: N=9, F (1, 44)=4.752, p=0.0347, and P30-80 (C), WT-Vehicle: N=14; Syngap1^{+/-}-Vehicle: N=14; WT-6BIO: N=16; Syngap1^{+/-}-6BIO: N=16, F (1, 56)=0.4686, p=0.4965, in accordance with an embodiment of the present disclosure. In FIG. 2, F refers to the fraction followed by the two sets of degrees of freedom; one for the numerator and one for the denominator, further the symbol “*” denotes statistical significance or p-value wherein “*” denotes p<0.05, “**” denotes p<0.01, and “***” denotes p<0.001. Data analysis was performed using two-way ANOVA and Tukey's multiple comparisons test.

FIG. 3 depicts the effect of 6-BIO on the anxiety of the wild type and Syngap1^{+/-} mice. FIGS. 3(A), (B) and (C) depicts the number of entries in the centre as a measure of anxiety was grouped into 2(A) P10-16, the critical developmental period, 2(B) P10-80, critical period till adulthood, and 2(C) P30-80, adulthood respectively and shown as individual data points. The FIG. 3 depicts that for group P10-80 where behaviour was performed at P16, P42, and P80 (A), WT-Vehicle: N=20; Syngap1^{+/-}-Vehicle: N=11; WT-6BIO: N=16; Syngap1^{+/-}-6BIO: N=14, F (1, 57)=18.01, p<0.0001, P10-80 where behaviour was exhibited at P42 (B), WT-Vehicle: N=18; Syngap1^{+/-}-Vehicle: N=10; WT-6BIO: N=15; Syngap1^{+/-}-6BIO: N=11, F(1, 50)=2.194, p=0.1449, and P30-80 (C) WT-Vehicle: N=13; Syngap1^{+/-}-Vehicle: N=12; WT-6BIO: N=15; Syngap1^{+/-}-6BIO: N=18, F (1, 54)=6.002, p=0.0176, in accordance with an embodiment of the present disclosure. In FIG. 3, F refers to the fraction followed by the two sets of degrees of freedom; one for the numerator and one for the denominator and further, the symbol “*” denotes statistical significance or p-value wherein “*” denotes p<0.05, “**” denotes p<0.01, and “***” denotes p<0.001. Data analysis was performed using two-way ANOVA and Tukey's multiple comparisons test.

FIG. 4 depicts the effect of 6-BIO on hyperactivity and anxiety of the wild type and Syngap1^{+/-} mice when administered only in critical period P10-16 and measured at P80. FIG. 4(A) depicts the total distance travelled and the data points correspond to WT-Vehicle: N=11; Syngap1^{+/-}-Vehicle: N=9; WT-6BIO: N=12; Syngap1^{+/-}-6BIO: N=13, F (1, 41)=1.676, p=0.2027, and FIG. 4 (B) depicts the number of entries in the centre and the individual data points correspond to WT-Vehicle: N=12; Syngap1^{+/-}-Vehicle: N=11; WT-6BIO: N=12; Syngap1^{+/-}-6BIO: N=14, F(1,45)=0.8829, P=0.3524, in accordance with an embodiment of the present disclosure. In FIG. 4, F refers to the fraction followed by the two sets of degrees of freedom; one for the numerator and one for the denominator and further, the symbol “*” denotes statistical significance or p-value wherein “*” denotes p<0.05, “**” denotes p-value<0.01, and “***” denotes p-value <0.001. Data analysis was performed using two-way ANOVA, Tukey's multiple comparisons test, was plotted as grouped data with individual values on the graph.

FIG. 5 depicts the effect of 6-BIO on the memory of the wild type and Syngap1^{+/-} mice. FIGS. 5(A), (B) and (C) depicts the discrimination index between a familiar and a novel object as a measure of the ability to recognize a familiar object and the mice were grouped into 5(A) P10-16, the critical developmental period, 5(B) P10-80, critical period till adulthood, and 5(C) P30-80, adulthood respectively and shown as individual data points. FIG. 5(A) depicts P10-16 where behaviour was done at P80 and the individual data points plotted were WT-Vehicle: N=11; Syngap1^{+/-}-Vehicle: N=6; WT-6BIO: N=9; Syngap1^{+/-}-6BIO: N=14, F (1, 36)=14.76, p=0.0005, FIG. 5(B) depicts P10-80 where behaviour was done at P80 and individual data points depict WT-Vehicle: N=16; Syngap1^{+/-}-Vehicle: N=7; WT-6BIO: N=11; Syngap1^{+/-}-6BIO: N=10, F (1, 40)=1.982, p=0.1669, and FIG. 5(C) depicts P30-80 where behaviour was done at P80 and the individual data points correspond to WT-Vehicle: N=8; Syngap1^{+/-} Vehicle: N=7; WT-6BIO: N=9; Syngap1^{+/-}-6BIO: N=15, F (1, 35)=7.687, p=0.0089, in accordance with an embodiment of the present disclosure. In FIG. 5, F refers to the fraction followed by the two sets of degrees of freedom; one for the numerator and one for the denominator and further, the symbol “*” denotes statistical significance or p-value wherein “*” denotes p<0.05, “**” denotes p<0.01, and “***” denotes p<0.001. Data analysis was performed using two-way ANOVA, Tukey's multiple comparisons test, was plotted as grouped data with individual values on the graph.

FIG. 6 depicts the effect of 6-BIO on the social interaction of wild type and Syngap1^{+/-} mice. FIGS. 6(A), (B) and (C) depicts the time spent with an empty jar as compared to one other mouse (stranger 1) and mice were grouped into FIG. 6(A) P10-16, the critical developmental period, where behaviour was done at P80 and individual data points depict WT-Vehicle: N=12; Syngap1^{+/-}-Vehicle: N=10; WT-6BIO: N=12; Syngap1^{+/-}-6BIO: N=14, F (3, 88)=1.481, p=0.2252. In FIG. 6(B) P10-80, critical period till adulthood, and individual data points correspond to WT-Vehicle: N=9; Syngap1^{+/-}-Vehicle: N=6; WT-6BIO: N=10; Syngap1^{+/-}-6BIO: N=10. F(3,62)=3.722, p=0.0158, and the FIG. 6(C) depicts the mice in the age group P30-80, adulthood and the individual data point depict N=12; Syngap1^{+/-}-Vehicle: N=14; WT-6BIO: N=12; Syngap1^{+/-}-6BIO: N=18, F (3, 104)=2.909, p=0.0381, in accordance with an embodiment of the present disclosure. In FIG. 6, F refers to the fraction followed by the two sets of degrees of freedom; one for the numerator and one for the denominator and further, the symbol “*” denotes statistical significance or p-value wherein “*” denotes p<0.05, “**” denotes p-value <0.01, and “***” denotes p-value <0.001. Data analysis was performed using two-way ANOVA and Tukey's multiple comparisons test.

FIG. 7 depicts the effect of 6-BIO on the social preference of wild type and Syngap1^{+/-} mice. Social preference was evaluated as time spent with stranger 2 as compared to stranger 1, respectively. FIG. 7(A) depicts mice grouped P10-16, the critical developmental period, where behaviour was done at P80 and individual data points depict WT-Vehicle: N=12; Syngap1^{+/-}-Vehicle: N=10; WT-6BIO: N=12; Syngap1^{+/-}-6BIO: N=14, F (3, 88)=0.6549, p=0.5820, FIG. 7(B) illustrates mice grouped at P10-80 where behaviour was done at P80 and the individual data points represent WT-Vehicle: N=9; Syngap1^{+/-}-Vehicle: N=8; WT-6BIO: N=10; Syngap1^{+/-}-6BIO: N=10, F (3, 66)=2.692, p=0.0532, and FIG. 7(C) depicts the mice in the group P30-80 where behaviour was done at P80, and individual data points represent WT-Vehicle: N=12; Syngap1^{+/-} Vehicle: N=13; WT-6BIO: N=12; Syngap1^{+/-}-6BIO: N=18, F (3, 102)=2.616, p=0.0551, in accordance with an embodiment of the present disclosure. In FIG. 7, F refers to the fraction followed by the two sets of degrees of freedom; one for the numerator and one for the denominator and further, the symbol “*” denotes statistical significance or p-value wherein “*” denotes p<0.05, “**” denotes p-value <0.01, and “***” denotes p-value <0.001. Data analysis was performed using two-way ANOVA and Tukey's multiple comparisons test.

FIG. 8 depicts the effect of 6-BIO administration on fluoroethyl-based tonic-clonic seizure threshold when given after a critical period of development (P30-80). The individual data points in FIG. 8, corresponds to F(1,25)=3.354, p=0.0790, WT-Vehicle: N=9; Syngap1^{+/-}-Vehicle: N=7; WT-6-BIO: N=7; Syngap1^{+/-} 6-BIO: N=6. F refers to the fraction followed by the two sets of degrees of freedom; one for the numerator and one for the denominator and further, the symbol “*” denotes statistical significance or p-value wherein “*” denotes p<0.05, “**” denotes p<0.01, and “***” denotes p<0.001, in accordance with the embodiment of the present disclosure Data analysis was performed using two-way ANOVA and Tukey's multiple comparisons tests.

FIG. 9 depicts the effect of 6-BIO on the behavioural deficits caused by 1-methyl-4-phenyl-1,2,3,6-tetrahydropyridine (MPTP). FIG. 9(A) depicts the latency to fall of various cohorts namely Placebo, MPTP and MPTP and 6-BIO, (MPTP+Co) as assessed by rotarod test, FIG. 9(B) depicts the representative trajectory maps of all mentioned cohorts as analysed by open field test and the FIG. 9(C) depicts the periphery distance travelled by all indicated cohorts as assessed by open field test. The effect of 6-BIO on the Placebo, MPTP and MPTPC Post cohorts are represented in FIG. 9 (D), depicting the latency to fall off the cohorts from Placebo, MPTP and MPTPC. Post groups as assessed by rotarod test and FIG. 9(E) depict the periphery distance travelled by all indicated cohorts as assessed by the open field test. Both the rotarod and open field behaviour analyses were performed on day 13 or day 7 post MPTP or vehicle administrations. Both the rotarod and open field behaviour analyses were performed on day 13 or day 7 post MPTP/vehicle administrations. 6-BIO (5 mg/kg) was administrated either along with MPTP (MPTPCCo) or post 48 hours of MPTP administration (MPTPCPost), in accordance with an embodiment of the present disclosure. Data analysis was performed using one-way ANOVA and the post-hoc Bonferroni test. Scale bar: 50 mm. Error bars, mean+SEM ns-nonsignificant, −P<0.001.

FIG. 10 depicts the effect of 6-BIO and its derivatives 6-BIO, 6-MIO, compound 50 and compound 51 on the autophagy flux as a measure of the levels of autophagosomes and autolysosomes, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

The term “including” is used to mean “including but not limited to”. “Including” and “including but not limited to” are used interchangeably.

For the purposes of the present disclosure, the term “(2′Z, 3′E)-6-Bromoindirubin-3′-oxime” or “6-BIO” refers to a member of the class of bindoles that is indirubin substituted at position 6 by a bromo group and in which the keto group at position 3′ undergoes condensation with hydroxylamine to form the corresponding oxime. In the present disclosure, the compound “6-BIO” is used based on its known property as a glycogen synthase kinase 3B (GSK-3B) inhibitor.

In the present disclosure, the term “6-MIO”, “compound 50” and “compound 51” refers to synthesized derivatives of the compound “6-BIO”. The term “6-MIO” refers to the compound 6-methoxyindirubin oxime. The term “compound 50” refers to the compound (2Z,3E)-6′-bromo-3-(hydroxyimino)-[2,3′-biindolinylidene]-2′-one. The term “compound 51” refers to the compound (2Z,3E)-6′-bromo-3-((2-hydroxyethoxy)imino)-[2,3′-biindolinylidene]-2′-one.

The term “behavioural deficit” refers to the condition wherein there may be an impairment in the physical, emotional, motor, social, and cognitive functions of a patient suffering from a neurodevelopmental or neurodegenerative disorder, such that age-specific aspects of behaviour are lacking in an individual. Since growth and development may not be on target, there could be developmental delays in behaviour as well.

The term “MPTP” refers to “1-methyl-4-phenyl-1,2,3,6-tetrahydropyridine”, a prodrug to the neurotoxin MPP+, which in its active form causes permanent symptoms of Parkinson's disease by destroying dopaminergic neurons in the substantia nigra of the brain in a subject and may be used to mimic neurodegenerative disease in a subject.

The term “neurodevelopmental disorders” refers to disorders such as autism spectrum disorder, intellectual disability, pervasive neurodevelopmental disorder, attention deficit hyperactivity disorder, neonatal epilepsy, specific learning disorders, communication disorders, speech disorder, Schizophrenia, and developmental language disorder wherein the development of the nervous system is affected, leading to abnormal brain function which results in deficits in emotional distress, social dysfunction, physical dysfunction, epileptic seizures, learning ability, self-control, and memory.

The term “neurodegenerative disorders” refers to disorders such as Parkinson's disease, Alzheimer's disease, Huntington's disease, ataxia, cerebral palsy, multiple sclerosis, dementia, amyotrophic lateral sclerosis, and Batten disease, wherein the nerve cells in the brain or peripheral nervous system lose function over time and ultimately die.

The term “SYNGAP1^{+/-} “refers to the mutated or wild type SYNGAP1 gene wherein the wild type SYNGAP1 gene i.e., SYNGAP1^{+/+} is translated into a functional SYNGAP1 protein and the mutated SYNGAP1 gene i.e., SYNGAP1^{-/+} translates to a non-functional SYNGAP1 protein.

In the present disclosure, the term “P(X−Y)” is used for referring to a developmental period of a subject from day X to day Y. The term “P” stands for post-natal, and the number denotes days after birth. For example; the term “P(10-16)” refers to the age group of mice subjects taken during the critical period of neurodevelopment, including 10-16 days old mice. Similarly, the term “P(10-80)” refers to the age of mice from the critical period of development to adulthood in subjects including subjects aged 10 days old to 80 days old. The term “P(30-80)” refers to the age group of subjects in their adulthood including subjects aged 30 day to 80 day old.

The term “subject” refers to any vertebrate animal and does not merely cover human or mice. Wild type (WT) mice, Syngap1^{+/-} mice and MPTP mice subjects have been used to exemplify the invention but, said exemplification should not be considered in any way limiting to the scope of the subject matter as covered under the term subject.

As illustrated in the background, current therapeutics are targeted towards alleviating the symptoms of neurological disorders rather than correcting the phenotypes developed in the neurodevelopmental and neurodegenerative disorders, especially after complete brain development in the subject. The most strategic therapies target the most commonly observed symptoms such as seizures, hypotonia, sleeplessness, mania, euphoria, anxiety and other social behaviours.

Cholesterol-lowering statins, such as lovastatin or atorvastatin, have been proven to ameliorate the deficits in social interactive behaviour and cognitive behaviour, in a 31-year-old patient as well as a 2-year-old child, respectively. However, pre-clinical studies have shown high dosage of atorvastatin for the long-term could cause significant deficits in cognitive capabilities and necessary exploratory behaviour. Patients below the age of 10 years, who have autism spectrum disorder, often also exhibit symptoms of psychiatric disorders that can be treated using lithium or its derivatives that primarily alleviate these symptoms. In human clinical trials, lithium has proven to be the potent drug for ameliorating mood disorders commonly found in neurodevelopmental disorders. Pre-clinical model-based studies have elucidated that the chronic usage of lithium is capable of revamping several aspects of synaptic plasticity that may be lost in neurological disorders, however, lithium toxicity causes several side effects such as vomiting, tremor or fatigue, enuresis or irritability in patients with an autism spectrum disorder. The increased dosage might also cause acute renal failure, seizures, altered mental status, confusion and coma.

Similarly, for neurodegenerative disorders as severe as Parkinson's disease, there is an unmet therapeutic intervention. Some medical options are available to alleviate the muscular symptoms, such as levodopa-carbidopa. Although levodopa is a precursor to dopamine with antiparkinsonian properties, the drug causes side effects such as nausea, vomiting, dizziness, loss of appetite, and weight loss. Other pharmaceutical interventions that can be taken along with levodopa-carbidopa options include catechol-O-methyltransferase (COMT) inhibitors, dopamine agonists and monoamine oxide B inhibitors. However, these drugs have severe side effects and are not advised for chronic usage. (Pubchem.ncbi.nlm.nih.gov. 2021. Levodopa. [online] Available at: <https://pubchem.ncbi.nlm.nih.gov/compound/levodopa>[Accessed 25 Feb. 2021]).

In order to overcome the limitations associated with the conventional treatment options available in the art, the present disclosure provides a GSK-3B inhibitor, 6-BIO. In particular, the present disclosure discloses the use of 6-BIO and its derivatives therapeutic in treating neurodevelopmental and neurodegenerative disorders. 6-BIO not only play a major role in alleviating symptoms associated with these disorders but also plays an important role in correcting the phenotype during the neurodevelopmental period. Thus, 6-BIO proves to be an effective and safe molecule for treating neurological disorders. Moreover, the derivatives of 6-BIO have been shown to be more efficacious in treating neurological disorders due to improved solubility and bioavailability.

One of the major causes of neurodevelopmental disorders is heterozygous mutations in the SYNGAP1 gene, leading to loss of function of SYNGAP1 protein. This protein causes aberrant maturation of dendritic spines resulting in anomalous excitation-inhibition (E/I) balance, which limits the normal progression and refinement of synaptic connections during the critical period of development in Syngap1^{+/-} subject. Since the current therapeutic options are related to alleviating the symptoms, rather than correcting the phenotype after a neurodevelopmental period, therefore, to cure this deficiency, the present disclosure provides the use of GSK-3B inhibitor, 6-BIO that corrects synaptic deficits in the long term potential and the electric potential of the receptor of the gamma-aminobutyric acid (EGABA) in young adolescent subjects after crossing the blood-brain barrier.

Several neurodegenerative disorders, such as Parkinson's disease are marked by the accumulation of misfolded proteins and dysfunctional mitochondria. The misfolded proteins aggregate and cause functional disruptions in the processes of chaperones, proteasome, or macroautophagy results in neuron death and eventually neurological degeneration. The 6-BIO or its derivative used in the present disclosure clear protein aggregates by toxic proteins such as the protein aggregates expressed from the mutated alpha-synuclein gene that causes Parkinson's disease and restores cellular homeostasis by inducing autophagy and strongly driving the autophagy flux resulting in aggregate clearance. The modulation of autophagy flux by 6-BIO is based on its inhibition of GSK3β activity.

In an embodiment of the present disclosure, there is provided with a composition comprising (2′Z, 3′E)-6-Bromoindirubin-3′-oxime or its derivative for the treatment of neurodevelopmental disorders and at least one excipient.

In an embodiment of the present disclosure, there is provided with a composition comprising (2′Z, 3′E)-6-Bromoindirubin-3′-oxime or its derivative for the treatment of neurodegenerative disorders and at least one excipient.

In an embodiment of the present disclosure, there is provided with a composition comprising (2′Z, 3′E)-6-Bromoindirubin-3′-oxime or its derivative for the treatment of neurodevelopmental disorders and at least one excipient, wherein the excipient is selected from the group consisting of buffers and solubilizing agents. In another embodiment of the present disclosure, the excipient is solubilizing agents, wherein the solubilizing agent is DMSO.

In an embodiment of the present disclosure, there is provided with a composition comprising (2′Z, 3′E)-6-Bromoindirubin-3′-oxime or its derivative for the treatment of neurodegenerative disorders and at least one excipient, wherein the excipient is selected from the group consisting of buffers and solubilizing agents. In another embodiment of the present disclosure, the excipient is solubilizing agents, wherein the solubilizing agent is DMSO.

In an embodiment of the present disclosure, there is provided with a composition comprising (2′Z, 3′E)-6-Bromoindirubin-3′-oxime or its derivative for the treatment of neurodevelopmental disorders and at least one excipient, wherein the neurodevelopmental disorder is selected from the group consisting of Autism spectrum disorder, Intellectual disability, Pervasive Neurodevelopmental Disorder or Attention Deficit Hyperactivity Disorder, Specific Learning Disorders, Communication Disorders, epileptic seizures, Schizophrenia, Speech disorder, and Developmental Language Disorder.

In an embodiment of the present disclosure, there is provided with a composition comprising (2′Z, 3′E)-6-Bromoindirubin-3′-oxime or its derivative for the treatment of neurodegenerative disorders and at least one excipient, wherein the neurodegenerative disorder is selected from the group consisting of Parkinson's disease, Alzheimer's disease, Huntington's Disease, Ataxia, Cerebral Palsy, Multiple Sclerosis, Dementia, Amyotrophic lateral sclerosis, and Batten disease.

In an embodiment of the present disclosure, there is provided with a composition comprising (2′Z, 3′E)-6-Bromoindirubin-3′-oxime or its derivative for the treatment of neurodevelopmental disorders and at least one excipient, wherein the excipient is selected from the group consisting of buffers and solubilizing agents, and wherein the neurodevelopmental disorder is selected from the group consisting of Autism spectrum disorder, Intellectual disability, Pervasive Neurodevelopmental Disorder, Attention Deficit Hyperactivity Disorder, Specific Learning Disorders, Communication Disorders, Speech disorder, and Developmental Language Disorder.

In an embodiment of the present disclosure, there is provided with a composition comprising (2′Z, 3′E)-6-Bromoindirubin-3′-oxime or its derivative for the treatment of neurodegenerative disorders and at least one excipient, wherein the excipient is selected from the group consisting of buffers and solubilizing agents, and wherein the neurodegenerative disorder in a subject is selected from the group consisting of Parkinson's disease, Alzheimer's disease, Huntington's Disease, Ataxia, Cerebral Palsy, Multiple Sclerosis or Dementia, Amyotrophic lateral sclerosis, and Batten disease.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene and wherein the behavioural deficits corrected by (2′Z, 3′E)-6-Bromoindirubin-3′-oxime or its derivative include hyperactivity, anxiety, novel object recognition, social interaction and social preferences.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene and wherein the behavioural deficits corrected by (2′Z, 3′E)-6-Bromoindirubin-3′-oxime or its derivative include hyperactivity, anxiety, novel object recognition, social interaction and social preferences.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene and wherein the subject is administered 1-10 mg/kg of (2′Z, 3′E)-6-Bromoindirubin-3′-oxime or its derivative per body weight of the subject.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene and wherein the subject is administered 1-10 mg/kg of (2′Z, 3′E)-6-Bromoindirubin-3′-oxime or its derivative per body weight of the subject.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene, and wherein the behavioural deficits corrected by (2′Z, 3′E)-6-Bromoindirubin-3′-oxime or its derivative is selected from the group consisting of hyperactivity, anxiety, novel object recognition, social interaction and social preferences, and wherein the subject is administered 1-10 mg/kg of (2′Z, 3′E)-6-Bromoindirubin-3′-oxime or its derivative per body weight of the subject.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene, and wherein the behavioural deficits corrected by (2′Z, 3′E)-6-Bromoindirubin-3′-oxime or its derivative are selected from the group consisting of hyperactivity, anxiety, novel object recognition, social interaction and social preferences and wherein the subject is administered 1-10 mg/kg of (2′Z, 3′E)-6-Bromoindirubin-3′-oxime or its derivative per body weight of the subject.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative, wherein the neurodevelopmental disorder is selected from the group consisting of Autism spectrum disorder, Intellectual disability, Pervasive Neurodevelopmental Disorder, Attention Deficit Hyperactivity Disorder, Specific Learning Disorders, Communication Disorders, Speech disorder, and Developmental Language Disorder.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative, wherein the neurodegenerative disorder is selected from the group consisting of Parkinson's disease, Alzheimer's disease, Huntington's Disease, Ataxia, Cerebral Palsy, Multiple Sclerosis, Dementia, Amyotrophic lateral sclerosis, and Batten disease.

In an embodiment of the present disclosure, there is provided with a method for correcting the behavioural deficits in a subject with neurodevelopmental disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene, and wherein the neurodevelopmental disorder is selected from the group consisting of Autism spectrum disorder, Intellectual disability, Pervasive Neurodevelopmental Disorder, Attention Deficit Hyperactivity Disorder, Specific Learning Disorders, Communication Disorders, Speech disorder, and Developmental Language Disorder.

In an embodiment of the present disclosure, there is provided with a method for correcting the behavioural deficits in a subject with neurodegenerative disorders in need thereof comprising administering to the subject a therapeutically effective amount of (2′Z, 3′E)-6-Bromoindirubin-3′-oxime or its derivative, wherein the behavioural deficits are caused by a mutation in Syngap1^{+/-} gene, and wherein the neurodegenerative disorder is selected from the group consisting of Parkinson's disease, Alzheimer's disease, Huntington's Disease, Ataxia, Cerebral Palsy, Multiple Sclerosis, Dementia, Amyotrophic lateral sclerosis, and Batten disease.

In an embodiment of the present disclosure, there is provided a method for correcting the behavioral deficits in a subject with neurodegenerative disorders in need thereof comprising administering to the subject a therapeutically effective amount of (2′Z, 3′E)-6-Bromoindirubin-3′-oxime or its derivative, wherein the behavioral deficits are caused by mutation in Syngap1^{+/-} gene, and wherein the behavioral deficits corrected by (2′Z, 3′E)-6-Bromoindirubin-3′-oxime or its derivative are selected from the group consisting of hyperactivity, anxiety, novel object recognition, social interaction, and social preferences, and wherein the subject is administered 1-10 mg/kg of (2′Z, 3′E)-6-Bromoindirubin-3′-oxime or its derivative per bodyweight of the subject, and wherein the neurodegenerative disorder is selected from the group consisting of Parkinson's disease, Alzheimer's disease, Huntington's Disease, Ataxia, Cerebral Palsy, Multiple Sclerosis, Dementia, Amyotrophic lateral sclerosis, and Batten disease.

In an embodiment of the present disclosure, there is provided a method for correcting the behavioral deficits in a subject with neurodevelopmental disorders in need thereof comprising administering to the subject a therapeutically effective amount of (2′Z, 3′E)-6-Bromoindirubin-3′-oxime or its derivative, wherein the behavioral deficits are caused by mutation in Syngap1^{+/-} gene, and wherein the behavioral deficits corrected by (2′Z, 3′E)-6-Bromoindirubin-3′-oxime or its derivative include hyperactivity, anxiety, novel object recognition, social interaction and social preferences, and wherein the subject is administered 1-10 mg/kg of (2′Z, 3′E)-6-Bromoindirubin-3′-oxime or its derivative per bodyweight of the subject, and wherein the neurodevelopmental disorder is selected from the group consisting of Autism spectrum disorder, Intellectual disability, Pervasive Neurodevelopmental Disorder, Attention Deficit Hyperactivity Disorder, Specific Learning Disorders, Communication Disorders, Speech disorder, and Developmental Language Disorder.

In an embodiment of the present disclosure, there is provided with a method for correcting the behavioural deficits in a subject with neurodevelopmental disorders in need thereof comprising administering to the subject a therapeutically effective amount of the composition as described herein.

In an embodiment of the present disclosure, there is provided with a method for correcting the behavioural deficits in a subject with neurodegenerative disorders in need thereof comprising administering to the subject a therapeutically effective amount of the composition as described herein.

In an embodiment of the present disclosure, there is provided a use of the (2′Z, 3′E)-6-Bromoindirubin-3′-oxime or its derivative for the treatment of neurodevelopmental disorders.

In an embodiment of the present disclosure, there is provided a use of the composition as described herein for the treatment of neurodevelopmental disorders.

In an embodiment of the present disclosure, there is provides a use of the (2′Z, 3′E)-6-Bromoindirubin-3′-oxime or its derivative for the treatment of neurodegenerative disorders.

In an embodiment of the present disclosure, there is provided with a use of the composition as described herein for the treatment of neurodegenerative disorders.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

The working and non-working examples as depicted in the forthcoming sections highlight the criticality of the use of the GSK-3B inhibitor 6-BIO and its derivatives in the correction of phenotype, notably social novelty, anxiety, and memory during the critical period but also in young adolescent model mice. It is further specified that the presence of 6-BIO is critical to alleviate the symptoms and to correct the phenotype, thus making 6-BIO and its derivative an effective molecule for treating neurodevelopmental and neurodegenerative disorders.

### Materials and Methods

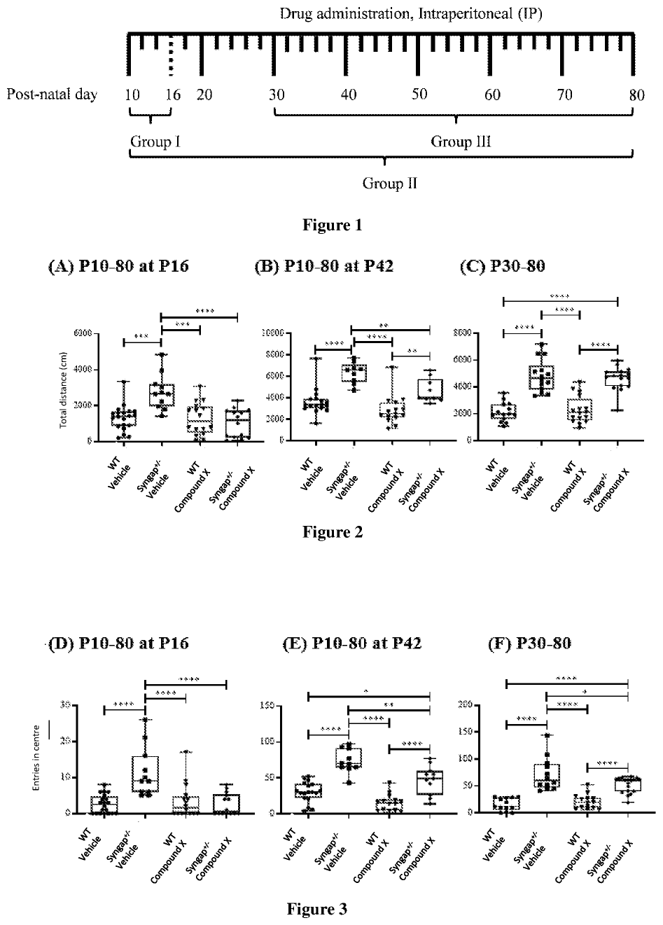

The ability of 6-BIO on the behavioural deficits related to neurodevelopmental disorders was observed based on its theoretical ability to correct synaptic plasticity, EGABA, and behavioural dysfunction in mice with mutated Syngap1 gene. Two populations of mice tested were: wild type (WT) mice and mice with mutated Syngap1 gene referred to as Syngap1^{+/-} mice. The behaviour was observed in two populations of mice in three different age groups based on the number of days, comprising Group I (P10-16), the critical developmental period, Group II: P (10-80), critical period till adulthood, and Group III (P30-80), comprising adult mice, wherein the adult mice were of 30 to 80 days old. In the control group and the Syngap1^{+/-} mice, the subjects were administered with 5 mg/kg of 6-BIO based on the bodyweight of the subject. The intraperitoneal injection schedule for 6-BIO (5 mg/kg) and age group segregation is shown in FIG. 1. The behaviour was observed at a standard age group for the groups I, II, and III at 16 days, 42 days and 80 days, respectively. For example, 2-9, male mice were used.

### Example 1

### Effect of 6-BIO on the Hyperactivity and Anxiety of the Syngap1^{+/-} Mice

Open field test: In the open field test, the hyperactivity or motor function, as well as the level of anxiety displayed by the mice, was observed in the wild type, Syngap1^{+/-} mice and Syngap1^{+/-} mice administered with 6-BIO. Syngap1^{+/-} mice in groups I, II and III. Mice were injected during the critical developmental period of P10-16 and behaviour was recorded at 16 days and 80 days, wherein mice were allowed to explore the open field chamber for 15-minutes and the total distance travelled by the mice was measured.

### Results

Hyperactivity: The results of the open field test are depicted in FIG. 2 and FIG. 3. In FIG. 2, the total distance travelled by the mice in the open field chamber was grouped and depicted as individual data points for P10-80 (critical period of development till adulthood) where behaviour was done at P16. In the three groups, results indicate that 6-BIO treated Syngap1^{+/-} mice (P10-16) showed partial correction of hyperactivity (total distance; behaviour at P80; Extended). However, hyperactivity was corrected when tested at P16 (P10-80 age group; FIG. 2A), but the partial correction was observed when performed at P42 (FIG. 2B) and no rescue behaviour at P30-80 (adulthood) (FIG. 2C), suggesting that the 6-BIO is most effective in correcting the phenotype when administered only during the critical period of development, as a full restoration of the phenotype was observed in 6-BIO treated Syngap1^{+/-} mice. Moreover, it is pertinent to note that the phenotypic levels observed in 6-BIO treated Syngap1^{+/-} mice were comparable to the phenotypic levels observed in the wild type mice.

Anxiety: The anxiety of the mice was observed based on the number of events of the mice entering the centre of the open-field arena. The results demonstrated that anxiety was corrected when behaviour was performed at P16 (FIG. 3(A)), P42 (FIG. 3(B)), P10-16 (critical period) age group (behaviour at P80), and a partial correction of the anxiety were performed between P30-80 (adulthood) (FIG. 3(C)) age groups, suggesting that anxiety was corrected when 6-BIO was administered after the critical period of development. These results indicate that 6-BIO restored the anxiety deficits observed in Syngap1^{+/-} mice. It can also be observed that the level of anxiety displayed in the 6-BIO treated Syngap1^{+/-} mice is comparable to the anxiety level observed in wild type mice, which is irrespective of the developmental period in which 6-BIO was administered.

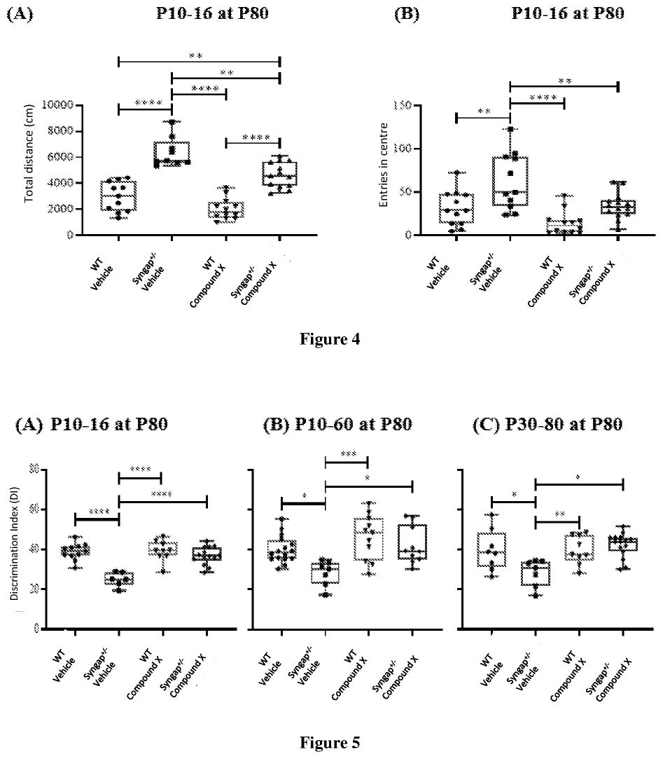

In FIGS. 4, (A) and (B) the data clearly illustrates that 6-BIO ameliorates hyperactivity and anxiety when administered only in critical period P(10-16), wherein the behaviour was observed or recorded at P80. This demonstrates that administration of 6-BIO only during the critical period of development is sufficient to restore the behavioural deficits observed in Syngap1^{+/-} mice.

It can be inferred from the above results that 6-BIO can be used for correcting deficits (hyperactivity, anxiety, etc.) in the subject. Owing to the properties of 6-BIO for correcting the behavioural deficits in the subject, 6-BIO can be used in the composition. It can be contemplated that a person skilled in the art can practice the same experiment as mentioned above for using the composition comprising 6-BIO or its derivative for correcting behavioural deficits in the subject.

### Example 2

### Effect of 6-BIO on the Memory of the Syngap1^{+/-} Mice

Novel object recognition test: The effect of 6-BIO on memory was exhibited, based on a novel object recognition test. The wild type and Syngap1^{+/-} mice in the absence of any treatment were compared with the mice intra-peritoneally injected with 6-BIO and their behaviour was recorded with regards to recognition of a familiar object versus reaction to a novel object.

### Results

The results indicated that the behaviour was performed in all three age groups of mice at 80 days (group I, group II, and group III) as shown in FIG. 5. FIG. 5 illustrates the results of group I (P10-16) in FIG. 4(A), group II (P10-80) in FIG. 5(B) and group III (P30-80) in FIG. 5(C) and the discrimination index was calculated. The results indicated that 6-BIO substantially corrected memory deficits in Syngap1^{+/-} mice. These results illustrate that 6-BIO, especially when administered after the critical period of development, corrected the memory deficits in Syngap1^{+/-} mice. These results in terms of memory levels were comparable to the memory levels observed in wild type mice. A person skilled in the art can use 6-BIO in the form of a composition, wherein the composition can be intra-peritoneally injected into a mouse. It can be appreciated that the composition comprising 6-BIO can be used for correcting the memory deficits in Syngap1^{+/-} mice.

### Example 3

### Effect of 6-BIO on the Social Interaction and Preferences of the Syngap1^{+/-} Mice

Social Novelty Test: Similar to the novel object recognition in Example 2, the social novelty test was performed for all age groups at 80 days. The social novelty test was performed based on the social interaction (SI) and social preference (SP) of the control wild type and Syngap1^{+/-} mice and wild type and Syngap1^{+/-} treated with 6-BIO. The social interaction was observed based on the time spent by the mice in isolation in comparison to time spent with one other mouse (stranger 1) and the social preference was observed based on the time spent with two different mice (stranger 1 and stranger 2)

### Results

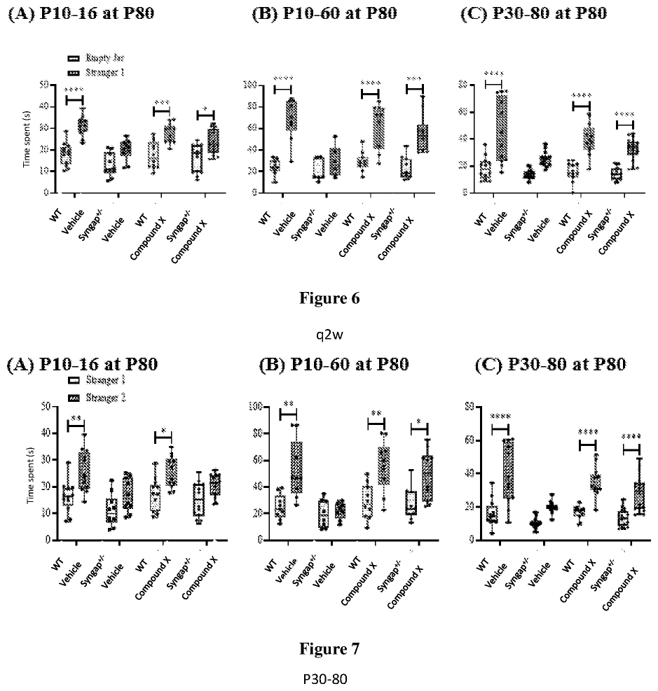

Social interaction (SI): The observations of the social interaction are illustrated in FIG. 6, wherein FIG. 6(A) depicts the results of group I (P10-16), FIG. 6(B) depicts the results of group II (P10-80) and FIG. 6(C) depicts the results of group III (P30-80). The results of all the groups were recorded at P80(80 days). The results indicated that the time spent with one other mouse (stranger 1) as compared to the empty jar for 6-BIO treated Syngap1^{+/-} mice were comparable to wild type mice in all age groups. The results further implied that Syngap1^{+/-} mice prefer object over other mice but this defect was corrected in 6-BIO administered Syngap1^{+/-} mice.

Social preference (SP): Similar to FIG. 6, FIG. 7 of the present disclosure illustrates the results of the social preference of the mice wherein FIG. 7(A) depicts the results of group I (P10-16), FIG. 7(B) depicts the results of group II (P10-80) and FIG. 7(C) depicts the results of group III (P30-80). The results of all the groups were recorded at P80 (80 days). The results indicated a lack of improvement in the social preference in 6-BIO treated Syngap1^{+/-} mice in group I (P10-16). Further, it can be observed that Syngap1^{+/-} mice prefer not to interact with a new mouse (stranger 2) and prefer a familiar mouse (stranger 1) but this defect was corrected in 6-BIO administered mice.

The results demonstrate that administration of 6-BIO restores social interaction deficits observed in Syngap1^{+/-} mice. Similarly, 6-BIO corrected social preference issues observed in Syngap1^{+/-} mice irrespective of the developmental time point of the administration of 6-BIO. Thus, it can be inferred from the results that the GSK-3B inhibitor, 6-BIO is effective in correcting the social interaction and preference deficits when administered after a critical period of development in Syngap1^{+/-} mice.

### Example 4

### Effect of 6-BIO on the Seizure Threshold on the Syngap1^{+/-} Mice

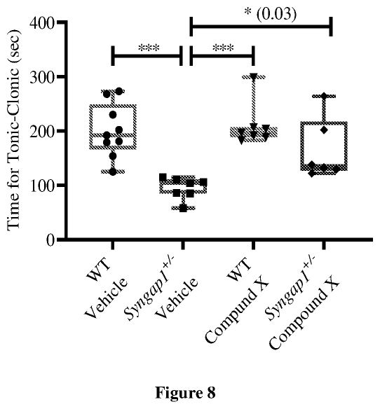

For investigating the effect of 6-BIO on the wild type and Syngap1^{+/-} mice in group III (P30-P80), mice were injected intraperitoneally with 6-BIO. The subsequent fluoroethyl-based seizure threshold i.e., susceptibility of mice to epileptic seizures was evaluated and plotted as grouped data showing individual points for the involuntary and rhythmic muscular contractions and relaxations referred to as clonus. The results as demonstrated in FIG. 8 depicts the effect of 6-BIO on the successive phases of tonic and clonic involuntary spasms experienced by the mice during a seizure, referred to as tonic-clonic. The results depicted that 6-BIO corrected the seizure threshold in Syngap1^{+/-} mice as compared to the levels observed in wild type mice, in the age group III (P30-80), when administered after the critical period (P10-16) of development. The Syngap1^{+/-} mice administered with 6-BIO displayed a lower seizure threshold restored to the susceptibility to epileptic seizures displayed by wild-type mice. This data suggests that 6-BIO can be used as a treatment for epilepsy (neurodevelopmental disorder). It can also be appreciated that the composition comprising 6-BIO exhibits similar results in terms of treating epilepsy in the subject.

### Example 5

### Effect of 6-BIO and Derivatives Thereof on Neurodegenerative Disorder

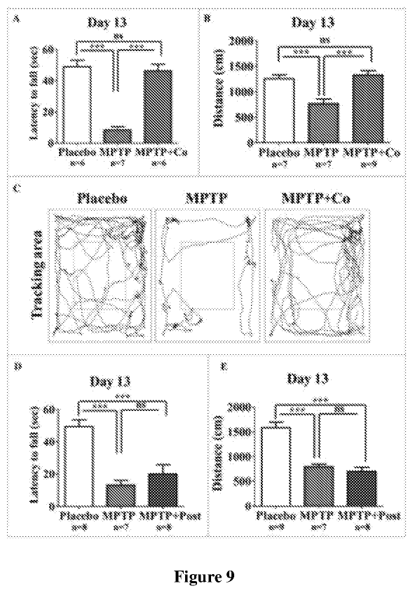

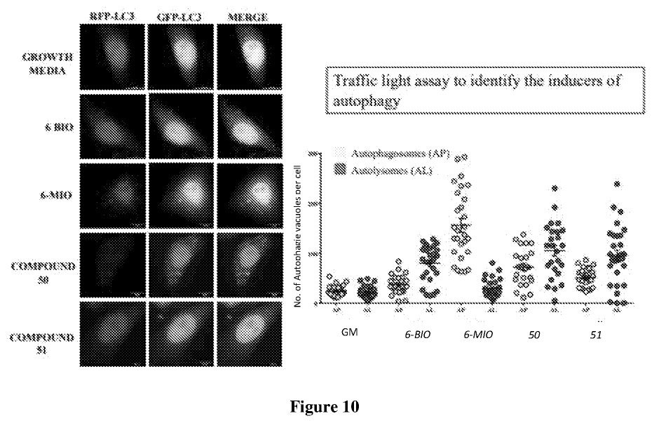

The effect of 6-BIO on neurodegenerative disorders such as Parkinson's was evaluated based on testing the ability of 6-BIO on toxic protein aggregates such as the SNCA/a-synuclein protein in mice (FIG. 9) and the modulation of the autophagy flux (FIG. 10). MPTP is a widely accepted mouse model to study Parkinsonism. It is known that the mice model of Parkinson's disease displays motor dysfunction Rotarod and open field test was used for estimation of the effect of 6-BIO on the mice model. For the study, mice were intraperitoneally injected with 6-BIO at a concentration of 5 mg/kg based on the body weight of the mice.

### Results

FIG. 9 and FIG. 10 illustrated the results observed on the effect of 6-BIO and its derivatives on neurodegenerative disorders. FIG. 9 depicts the effect of administration of 5 mg/kg of 6-BIO on (A) the latency to fall of various cohorts namely placebo mice, MPTP mice (Parkinsonian mice) and the MPTP mice administered with 6-BIO (MPTP+Co) was assessed by rotarod test (B) Representative trajectory maps of all mentioned cohorts as analysed by open field test. (C) Periphery distance travelled by all indicated cohorts as assessed by open field test. The effect of 6-BIO (5 mg/kg) on various cohorts namely placebo mice, mice administered with MPTP and mice administered with 5 mg/kg of 6-BIO post 48 hours of administration MPTP (MPTPCPost) were depicted in FIG. 9 (D) latency to fall of various cohorts namely Placebo, MPTP and MPTPCPost as assessed by rotarod test and FIG. 9 (E) based on the periphery distance travelled by all indicated cohorts as assessed by the open field test. Both the rotarod and open field behaviour analyses were performed on day 13 or day 7 post MPTP or vehicle administrations. The administration of 5 mg/kg of 6-BIO was either along with MPTP (MPTPCCo) or post 48 hours of MPTP administration (MPTPCPost). Statistical analysis was performed using one-way ANOVA and the post-hoc Bonferroni test. Scale bar: 50 mm. Error bars, mean § SEM ns-nonsignificant, −P<0.001. The results demonstrate that 6-BIO when administered along with MPTP, protects neurons from degeneration. Thus, 6-BIO is a potent molecule to treat neurodegeneration.

The derivatives of 6-BIO such as the compound 6-methoxyindirubin oxime (6-MIO), compound 50 and compound 51 were synthesized to improve their water solubility. The effect of 6-BIO and the aforementioned derivatives of 6-BIO on autophagy flux in the form of autophagosomes and autolysosomes are depicted in FIG. 10. This result illustrates that 6-MIO (derivative) increased both the autophagosomes and autolysosomes and compound 50 and compound 51 increased the levels of autolysosome. Analysis using the parent molecule (6-BIO), negative control and a growth medium (GM) and the derivatives of 6-BIO indicates that the majority of the derivatives show better efficacy than 6-BIO. Thus, the derivatives have a better potency to restore neuronal function to wild type level in neurodevelopmental disorders and play a neuroprotective role in neurodegeneration.

These results indicate that 6-BIO and the derivatives thereof such as 6-MIO, compound 50 and compound 51 increased the levels of autophagosomes and autolysosomes in cells as compared to the growth media control. These results suggest that 6-BIO increases the autophagic flux.

Overall, the use of 6-BIO and the composition comprising 6-BIO in the restoration of synaptic function and plasticity is disclosed in the present disclosure at a concentration of 5 mg/kg based on the weight of the mice. The wild type and Syngap1^{+/-} mice were divided into three major age groups Group I: P10-16 (critical period), Group II: P10-80 (critical period till adulthood), and Group III: P30-80 (adulthood) as shown in FIG. 1. The criticality of the present disclosure lies in the restoration of the synaptic function, plasticity, and EGABA by the correction of behavioural deficits such as social novelty, hyperactivity, anxiety, and memory, not only when 6-BIO was administered during the critical period (P10-16) but also in young adolescent Syngap1^{+/-} mice. These results are summarised in Table 1. The results indicated that intraperitoneal injection of 6-BIO or composition comprising 6-BIO normalized an altered Excitation/Inhibition balance and the deficits of synaptic transmission and behavioural performance like social novelty, anxiety and spatial memory.

TABLE 1

| | | Group (Age group in days) | | | |
|---|---|---|---|---|---|
| | | Group I | Group I | Group II | Group III |
| Recording day | | (P10-16) | (P10-16) | (P10-80) | (P30-80) |
| Experiment | Test | P16 | P80 | P42/P80 | P80 |
| Open field | Motor | Corrected | Partially | Partially | Partially |
| | function | | Corrected | Corrected | Corrected |
| | Anxiety | Corrected | Corrected | Partially | Partially |
| | | | | Corrected | Corrected |
| Novel Object | N/A | — | Corrected | Corrected | Corrected |
| Recognition | | | | | |
| Social | Interference | — | Corrected | Corrected | Corrected |
| | Preference | — | Not | Corrected | Corrected |
| | | | Corrected | | |
| Seizure | | — | — | — | Corrected |
| threshold | | | | | |
| Long term | — | — | — | Corrected | Corrected |
| potential | | | | | |
| E_{GABA}, | — | Corrected | — | — | — |
| Cl⁻ reversal | | | | | |
| potential | | | | | |

### Advantages of the Present Disclosure

The present disclosure discloses the use of 6-BIO for correcting the behavioural deficits as well as the excitation potential and synaptic plasticity in neurodevelopmental and neurodegenerative disorders. In particular, the present disclosure discloses a composition comprising 6-BIO or its derivative for treating neurodevelopmental and neurodegenerative disorders. The restoration of the deficits was inducible by the administration of 6-BIO not only during the critical period but also in young adolescent Syngap1^{+/-} mice. The results of the present disclosure support that the GABAergic circuit was disrupted during development and modulating this circuit by 6-BIO restored cognitive, emotional, and social symptoms that result from hard-wired neuronal circuit damage during development by late pharmacological intervention in adulthood.

1. A composition comprising (2′Z, 3′E)-6-Bromoindirubin-3′-oxime or its derivative for the treatment of neurodevelopmental disorders and at least one excipient.

2. A composition comprising (2′Z, 3′E)-6-Bromoindirubin-3′-oxime or its derivative for the treatment of neurodegenerative disorders and at least one excipient.

3. The composition as claimed in claim 1, wherein the excipient is selected from the group consisting of buffers, and solubilizing agents,

4. A method for correcting the behavioural deficits in a subject with neurodevelopmental disorders in need thereof comprising administering to the subject a therapeutically effective amount of (2′Z, 3′E)-6-Bromoindirubin-3′-oxime or its derivative.

5. A method for correcting the behavioural deficits in a subject with neurodegenerative disorders in need thereof comprising administering to the subject a therapeutically effective amount of (2′Z, 3′E)-6-Bromoindirubin-3′-oxime or its derivative.

6. The method as claimed in claim 4, for correcting the behavioural deficits caused by a mutation in Syngap1^{+/-} gene in a subject with neurodevelopmental disorders in need thereof comprising administering to the subject a therapeutically effective amount of (2′Z, 3′E)-6-Bromoindirubin-3′-oxime or its derivative.

7. The method as claimed in claim 5, for correcting the behavioural deficits caused by a mutation in Syngap1^{+/-} gene in a subject with neurodevelopmental disorders in need thereof comprising administering to the subject a therapeutically effective amount of (2′Z, 3′E)-6-Bromoindirubin-3′-oxime or its derivative.

8. The method as claimed in claim 4, wherein the behavioural deficits corrected by (2′Z, 3′E)-6-Bromoindirubin-3′-oxime or its derivative is selected from the group consisting of hyperactivity, anxiety, novel object recognition, social interaction and social preferences.

9. The method as claimed in claim 4, wherein the subject is administered 1-10 mg/kg of (2′Z, 3′E)-6-Bromoindirubin-3′-oxime or its derivative per body weight of the subject.

10. The composition as claimed in claim 1, wherein the neurodevelopmental disorder is selected from the group consisting of Autism spectrum disorder, Intellectual disability, Pervasive Neurodevelopmental Disorder, Attention Deficit Hyperactivity Disorder, Specific Learning Disorders, Communication Disorders, Epilepsy, Schizophrenia, Speech disorder, and Developmental Language Disorder.

11. The composition as claimed in claim 2, wherein the neurodegenerative disorder is selected from the group consisting of Parkinson's disease, Alzheimer's disease, Huntington's Disease, Ataxia, Cerebral Palsy, Multiple Sclerosis, Dementia, Amyotrophic lateral sclerosis, and Batten disease.

12. The method as claimed in claim 4, wherein the neurodevelopmental disorder is selected from the group consisting of Autism spectrum disorder, Intellectual disability, Pervasive Neurodevelopmental Disorder, Attention Deficit Hyperactivity Disorder, Specific Learning Disorders, Communication Disorders, Epilepsy, Schizophrenia, Speech disorder, and Developmental Language Disorder.

13. The method as claimed in claim 5, wherein the neurodegenerative disorder is selected from the group consisting of Parkinson's disease, Alzheimer's disease, Huntington's Disease, Ataxia, Cerebral Palsy, Multiple Sclerosis, Dementia, Amyotrophic lateral sclerosis, and Batten disease.
